(19) United States
(12) Patent Application Publication
(10) Pub. No.: US 2024/0226085 A1
(43) Pub. Date: Jul. 11, 2024

(54) KYNRENINE AND DERIVATIVES THEREOF FOR TREATING ATROPHIC SCARRING

(71) Applicant: BirchBioMed Inc., King City (CA)

(72) Inventors: Mark S. Miller, King City (CA); Jonathan W. Bourne, Fairport, NY (US)

(21) Appl. No.: 18/559,181

(22) PCT Filed: May 9, 2022
(86) PCT No.: PCT/CA22/50722

§ 371 Date: Nov. 6, 2023

Related U.S. Application Data
(60) Provisional application No. 63/185,994, filed on May 7, 2021.

(51) Int. Cl. A61K 31/4704; A61K 9/00; A61K 9/06; A61K 47/02; A61K 47/10; A61K 47/44; A61P 17/02

(52) U.S. Cl. CPC A61K 31/4704; A61K 9/0014; A61K 9/06; A61K 47/02; A61K 47/10; A61K 47/44; A61P 17/02

(57) ABSTRACT

This invention relates to the field of atrophic scarring and describes methods of using small molecule chemical compounds and formulations thereof to reduce the appearance of atrophic acne scarring. These methods and formulations are contemplated for cosmetic uses of reducing the appearance of acne scarring, or for potential therapeutic uses of reducing, reversing, or treating acne scarring. Also contemplated are use of these compounds to reduce the appearance of atrophic scarring or for potential therapeutic uses of reducing, reversing, or treating atrophic scarring.

### FIELD OF THE INVENTION

The present invention relates to methods of reducing the appearance of acne scarring on a person by applying or administering a small molecule chemical compound. Reducing the appearance of acne scarring is desirable for aesthetic purposes and cosmetic purposes, and methods and compositions to reduce the appearance of acne scarring would be commercially desirable to the cosmetic and pharmaceutical industries as well as to manufacturers of chemicals.

### BACKGROUND OF THE INVENTION

The global cosmetic industry is a multi-billion dollar market, and pharmaceutical sales for some skin conditions exceed a billion dollars per year (e.g., psoriasis). A common skin disease is acne vulgaris, often commonly referred to as acne, that according to Hession et al. (2015), “affecting nearly all adolescents and 12 to 51 percent of adults aged 20 to 49.” According to Shute, in 2013 the prevalence of acne in the US was approximately 50 million people and represented >$1 billion in treatment and treatment-related costs (2019). Other estimates place the global prevalence of acne at >9% (Heng & Chew, 2020) and some studies project the global market acne market to exceed 7 billion by 2025 (Duru & Orsal, 2021).

Acne vulgaris is a non-life threatening condition that can cause localized discomfort and scarring, and can lead to psychological complications (e.g., reduced self-esteem, body-image distortion) and psychiatric complications (e.g., anxiety or depression) (Duru & Orsal, 2021; Heng & Chew, 2020). Acne vulgaris is a multi-factorial process that is generally understood to arise from over-secretion by the sebaceous glands in the skin which lead to clogged psilosebaceous follicles of the face (Duru & Orsal, 2021) or face, chest, upper arms, and back (Connolly et al., 2017). According to Heng & Chew (2020), patients typically present with comedones (clogged follicles), papules (raised lesions), or postules (lesions filled with pus), and severe acne cases may present with nodules or cysts. A challenge with the diagnosis and treatment of acne is that dermatologists disagree on the minimal symptoms needed to formally diagnose as acne and there are now over 25 different systems used to grade severity of acne (Heng & Chew, 2020). And “[d]espite multiple ways to treat acne, no consensus exists on the best approach to acne management” (Duru & Orsal, 2021).

A common sequela of acne, especially acne vulgaris, is acne scarring (Eitta et al., 2019; Hession et al., 2015), with many patients having some degree of scarring (Connolly et al., 2017). Various treatment modalities for acne scars are known with varying degrees of success (Eitta et al., 2019; Hession et al., 2015). According to Eitta et al. (2019), “Post-acne scarring remains a common problem despite advances in the treatment of acne.”

Acne scarring is subdivided into two different types of scarring hypertrophic scarring and atrophic scarring (Connolly et al., 2017; Hession et al., 2015). The most common form of acne scarring is atrophic scars where there is a net destruction of collagen in the dermis, while much less common is hypertrophic or keloid acne scars where there is a net gain of collagen (Connolly et al., 2017). The atrophic scars are further morphologically sub-divided into boxcar, icepick, or rolling scar types (Connolly et al., 2017; Hession et al., 2015).

According to Connolly et al. (2017), boxcar scars represent about 20-30% of atrophic scars, and are wider “round-to-oval depressions with sharply demarcated vertical edges”, icepick scars are the most common (60-70%) and are narrow V-shaped epithelial tracts with sharp margins extending vertically to the deep dermis or subcutaneous tissue, while rolling scars represent about 15-25% of atrophic scars and causes an uneven skin surface that results in superficial shadowing and an undulating skin surface appearance. Treatments of atrophic scars include removing or damaging part of the skin, for example dermal abrasion to remove part of the epidermal (outer) layer of the skin or use of lasers to cause localized damage with the goal of stimulating dermal fibroblasts to replace the atrophied collagen and elastin by producing new material. Other treatment options include microneedling to induce new collagen deposition, subcission where a needle or other tool is used to injure a fibrous layer below the dermis and induce new collagen formation, and punch excision/elevation where a local area of atrophic scar is either removed and replaced with a graft or is cut and lifted to fill the atrophic scar depression at the skin surface and the void space is replaced by newly deposited collagen. In some instances, individual atrophic acne scars can also be treated with fillers where a material is injected into the skin to help fill the atrophic scar site (Connolly et al., (2017).

In light of high global prevalence of acne and the common subsequent acne scarring that results, new compositions and methods to reduce, reverse, or treat acne scarring or to reduce the appearance of acne scarring continue to be of commercial and industrial interest for both medical and cosmetic purposes.

### SUMMARY OF THE INVENTION

The present invention relates to formulations and methods of reducing or decreasing the appearance of atrophic scarring on a person by applying or administering a small molecule chemical compound. Acne and acne scarring are common problems, with atrophic scarring being the most common form of acne scarring. Reducing the appearance of atrophic acne scarring, or treatments to reduce atrophic acne scarring are desirable for cosmetic, aesthetic, and medical reasons.

In one embodiment, the present invention contemplates a method to reduce the appearance of acne scarring, atrophic acne scarring, or post-acne atrophic scarring comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to treat the appearance of acne scarring, atrophic acne scarring, or post-acne atrophic scarring comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to treat acne scarring, atrophic acne scarring, or post-acne atrophic scarring comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to reverse acne scarring, atrophic acne scarring, or post-acne atrophic scarring comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to prevent acne scarring, atrophic acne scarring, or post-acne atrophic scarring comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to conceal acne scarring, atrophic acne scarring, or post-acne atrophic scarring comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to treat, reverse, conceal, or reduce the appearance of atrophic scarring comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid. In one embodiment, the atrophic scarring is a presumed, likely, or confirmed sequelae of acne. In one embodiment, the atrophic scarring is a presumed or likely sequelae of acne vulgaris. In one embodiment, the atrophic scarring is a sequelae of acne vulgaris.

In one embodiment, the present invention contemplates a compound selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid, for use in the treatment of atrophic scarring. In one embodiment, the atrophic scarring is atrophic acne scarring. In one embodiment, the compound is kynurenic acid for use in the treatment of atrophic scarring. In one embodiment, the compound is kynurenic acid for use in the treatment of atrophic acne scarring. In one embodiment, kynurenic acid is a component of a composition formulated for topical administration for use in the treatment of atrophic scarring or atrophic acne scarring. In one embodiment, the compound is kynurenine for use in the treatment of atrophic scarring. In one embodiment, the compound is kynurenine for use in the treatment of atrophic acne scarring.

In one embodiment, the compound is a component of a composition formulated for injection and is administered by injection. In one embodiment the injection is administered as a subcutaneous, intradermal, or an intramuscular injection.

In one embodiment, the compound is a component of a composition formulated for oral delivery. In one embodiment the compound is an ingredient in a product intended for oral consumption.

In one embodiment, the compound is a component of a composition formulated for topical delivery. In one embodiment, the compound is a component of a composition formulated for topical application. In one embodiment the compound is an ingredient in a lotion, cream, gel, solution, or suspension intended for topical use. In one embodiment, the compound is 0.05 to 10% by weight of the lotion, cream, gel, solution, or suspension. In one embodiment, the compound is 0.05 to 6% by weight of the lotion, cream, gel, solution, or suspension. In one embodiment, the compound is 0.05 to 3% by weight of the lotion, cream, gel, solution, or suspension. In one embodiment, the compound is 0.1 to 1% by weight of the lotion, cream, gel, solution, or suspension. In one embodiment, the compound is 0.1 to 0.5% by weight of the lotion, cream, gel, solution, or suspension. In one embodiment, the compound is 0.25 to 0.5% by weight of the lotion, cream, gel, solution, or suspension. In one embodiment, the compound is kynurenic acid. In one embodiment the compound is kynurenine. In one embodiment, kynurenic acid is 0.1 to 1% by weight of the lotion, cream, gel, solution, or suspension. In one embodiment, kynurenic acid is 0.1 to 0.5% by weight of the lotion, cream, gel, solution, or suspension. In one embodiment, kynurenic acid is 0.5% by weight of the lotion, cream, gel, solution, or suspension. In one embodiment, kynurenic acid is 0.25% by weight of the lotion, cream, gel, solution, or suspension. In one embodiment, kynurenic acid is 0.1% by weight of the lotion, cream, gel, solution, or suspension.

In some embodiments, the application, administration, or use of the compound is contemplated as once, twice, or thrice daily.

### BRIEF DESCRIPTION OF THE DRAWINGS

FIG. 1A is a photographic representation of a target facial skin area of a female subject in their mid-40s with treatment-resistant stable atrophic scarring from acne before application of topical kynurenic acid (0.5% by weight) cream.

FIG. 1B is a photographic representation of the target facial skin area of the female subject of FIG. 1A after bidaily use of topical kynurenic acid (0.5% by weight) for 8 weeks. FIG. 1B shows significantly diminished appearance of atrophic scarring. The dashed black box in each image highlights the similar target area on the right cheek region in both images for comparison.

### DETAILED DESCRIPTION

### I. Definitions

There are provided herein a number of compounds for use in the treatment of atrophic acne scarring, or for the reduction in the appearance of atrophic acne scarring. In the context of the current description, the term ‘treatment’ may refer to treatment of existing atrophic acne scarring, or alternately may refer to treatment which occurs before atrophic acne scarring in order to prevent the development or progression of scarring. The compounds described herein may be in isolation, or may be linked to or in combination with tracer compounds, liposomes, carbohydrate carriers, polymeric carriers or other agents or excipients as will be apparent to one of skill in the art. In an alternate embodiment, such compounds may comprise a medicament, wherein such compounds may be present in a pharmacologically effective amount. The compounds may be suitable for administration to a subject in need thereof, by virtue of the fact that the subject may benefit from prophylaxis or treatment of atrophic acne scarring. The compounds may also include tautomers or stereoisomers.

As used herein, KA or KynA may be used as abbreviations for kynurenic acid (CAS #492-27-3) and XA may be used as an abbreviation for xanthurenic acid (CAS #59-00-7). L-kynurenine (CAS #2922-83-0) may be represented herein as L-Kyn and D-kynurenine (CAS #13441-51-5) may be represented herein as D-Kyn. Unless otherwise stated, kynurenine includes L-Kyn, D-Kyn, and a racemic mixture of the two (and where the racemic mixture may be represented as DL-Kyn or DL-Kynurenine, CAS #343-65-7). Stereochemistry of other amino acids may similarly be indicated as D-, L-, or DL- to refer to the racemic mixture thereof.

The term ‘medicament’ as used herein refers to a composition that may be administered to a patient or test subject and is capable of producing an effect in the patient or test subject. The effect may be chemical, biological or physical, and the patient or test subject may be human, or a non-human animal, such as a rodent or transgenic mouse, or a dog, cat, cow, sheep, horse, hamster, guinea pig, rabbit or pig. The medicament may be comprised of the effective chemical entity alone or in combination with a pharmaceutically acceptable excipient.

The term ‘pharmaceutically acceptable excipient’ may include any and all solvents, dispersion media, coatings, antibacterial, antimicrobial or antifungal agents, isotonic and absorption delaying agents, and the like that are physiologically compatible. An excipient may be suitable for intravenous, intraperitoneal, intramuscular, subcutaneous, intrathecal, topical or oral administration. An excipient may include sterile aqueous solutions or dispersions for extemporaneous preparation of sterile injectable solutions or dispersion. Use of such media for preparation of medicaments is known in the art.

Compounds or compositions according to some embodiments may be administered in any of a variety of known routes, or formulated for use in administration. Examples of methods that may be suitable for the administration of a compound include orally, intravenous, inhalation, intramuscular, subcutaneous, topical, intraperitoneal, intra-rectal or intra-vaginal suppository, sublingual, and the like. The compounds described herein may be administered as a sterile aqueous solution, or may be administered in a fat-soluble excipient, or in another solution, suspension, patch, tablet or paste format as is appropriate. Other methods known in the art for making formulations are found in, for example, “Remington's Pharmaceutical Sciences”, (19^{th} edition), ed. A. Gennaro, 1995, Mack Publishing Company, Easton, Pa.

The dosage of the compositions or compounds of some embodiments described herein may vary depending on the route of administration (oral, injection, topical, or the like) and the form in which the composition or compound is administered (solution, controlled release or the like). Determination of appropriate dosages is within the ability of one of skill in the art. As used herein, an ‘effective amount’, a ‘therapeutically effective amount’, or a ‘pharmacologically effective amount’ of a medicament refers to an amount of a medicament present in such a concentration to result in a therapeutic level of drug delivered over the term that the drug is used. This may be dependent on mode of delivery, time period of the dosage, age, weight, general health, sex and diet of the subject receiving the medicament. As used herein, an “effective amount” means the quantity necessary to render the necessary result. For example, an effective amount of a therapeutic is a level effective to treat, cure, or alleviate the symptoms of a disease for which the therapeutic agent is/are being administered. Methods of determining effective amounts are known in the art.

Any terms not directly defined herein shall be understood to have the meanings commonly associated with them as understood within the art of the invention. As employed throughout the specification, the following terms, unless otherwise indicated, shall be understood to have the following meanings.

### II. Biology

Scarring is a natural part of body's repair mechanism, and scarring in skin produces atrophic, hypertrophic, or keloid scars (Patel et al., 2014; Sitohang et al., 2021). Hypertrophic and keloid scars result in an excess deposition of collagen (Connolly, 2017), with hypertrophic scars contained within the wound area and keloid expanding beyond the original wound site. In contrast, atrophic scars are marked by a net loss in collagen and dermal depressions (Connolly et al., 2017; Nassar et al., 2020; Patel et al., 2014; Sitohang et al., 2021). According to Nassar et al. (2020), “[a]trophic scars are dermal depressions usually caused by collagen damage during tissue repair after surgery or trauma” (pg. 5) and “[a]trophic scarring results usually during wound healing with inadequate production of collagen” (pg. 1). Patel et al. (2014) states, “[a]trophic scars are defined histologically as scars showing a loss of collagen” (pg. 2).

Treatment of atrophic scars resulting from acne continues to be a challenge for dermatologists and treatment modalities are not standardized (Sitohang et al., 2021). Common treatments involve either stimulating the deposition of new collagen through techniques such as controlled injury to the tissue (e.g., microneedle, laser, chemical or mechanical dermal abrasion) or the addition of filler materials (Connolly et al., 2017; Hession et al., 2015; Patel et al., 2014).

Although it is not necessary to understand the mechanism of an invention and without being bound by any particular theory, the use of kynurenine and other kynurenine pathway metabolites, including kynurenic acid, have previously been described to treat fibroproliferative disorders, namely hypertrophic scarring and keloids (U.S. Pat. No. 9,737,523B2). Fibroproliferative disorders are marked by excessive accumulation of extracellular matrix, and kynurenines (including kynurenic acid) were shown to increase expression MMP-1 and MMP-3 enzymes in dermal fibroblasts ('523 FIGS. 2 and 3, respectively) and downregulate collagen 1 and fibronectin expression by dermal fibroblasts ('523 FIGS. 10 and 16, respectively). Topical application to skin tissue using a rabbit ear hypertrophic scarring model confirmed the dermal fibroblast results, showing that kynurenine decreased collagen and increased MMP-1 relative to controls ('523, FIG. 13). This combination of upregulating known matrix degrading enzymes (i.e., MMP-1 and MMP-3) with concomitant downregulation of collagen 1, as was shown in the '523 patent, effectively reverses hypertrophic scarring in an animal model and effectively reverses hypertrophic keloid scarring in skin of human subjects (BirchBioMed Inc., 2021). However, these data teach away from the use of kynurenines, including kynurenic acid, in treating atrophic scarring. Atrophic scarring is marked by a net loss (“atrophy”) of collagen and other matrix proteins while in contrast hypertrophic or keloid scarring represent the opposite with a net gain, typically excessive, (“hypertrophy”) of collagen and other matrix proteins. Upregulating known matrix degrading enzymes (i.e., MMP-1 and MMP-3) with concomitantly downregulating collagen 1 in skin, as was shown in the '523 patent, would accelerate a net loss of skin matrix and would be anticipated to potentiate local atrophy. Put simply, a person skilled in the art would reasonably expect increasing catabolic enzymes (i.e., MMP-1 and MMP-3) and decreasing matrix anabolic proteins (collagen 1) would exacerbate atrophic scarring.

### III. Methods of Compound Use & Formulations

In one embodiment, the present invention contemplates a method to reducing the appearance of acne scarring, atrophic acne scarring, or post-acne atrophic scarring comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to treat the appearance of acne scarring, atrophic acne scarring, or post-acne atrophic scarring comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to treat acne scarring, atrophic acne scarring, or post-acne atrophic scarring comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to reverse acne scarring, atrophic acne scarring, or post-acne atrophic scarring comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to prevent acne scarring, atrophic acne scarring, or post-acne atrophic scarring comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to conceal acne scarring, atrophic acne scarring, or post-acne atrophic scarring comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment the, the present invention contemplates a method to prevent acne scarring, atrophic acne scarring, or post-acne atrophic scarring comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

In one embodiment, the present invention contemplates a method to treat, reverse, conceal, or reduce the appearance of atrophic scarring comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid. In one embodiment, the atrophic scarring is a presumed, likely, or confirmed sequelae of acne. In one embodiment, the atrophic scarring is a presumed or likely sequelae of acne vulgaris. In one embodiment, the atrophic scarring is a sequelae of acne vulgaris.

In one embodiment the, the present invention contemplates a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid for the treatment of atrophic scarring, atrophic acne scarring, or post-acne atrophic scarring.

In one embodiment, the present invention contemplates a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid for the reduction in the appearance of atrophic scarring, atrophic acne scarring, or post-acne atrophic scarring.

In one embodiment, the present invention contemplates a compound selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid, for use in the treatment of atrophic scarring. In one embodiment, the atrophic scarring is atrophic acne scarring. In one embodiment, kynurenic acid for use in the treatment of atrophic scarring. In one embodiment, the compound is kynurenic acid for use in the treatment of atrophic acne scarring. In one embodiment, kynurenic acid is a component of a composition formulated for topical administration for use in the treatment of atrophic scarring or atrophic acne scarring. In one embodiment, kynurenic acid is a component of a composition formulated for topical administration for use in the concealment of atrophic scarring or atrophic acne scarring. In one embodiment, kynurenic acid is a component of a composition formulated for topical administration for use as a cosmetic treatment of atrophic scarring or atrophic acne scarring. In one embodiment, kynurenic acid is a component of a composition formulated for topical administration for use as a cosmetic product for reducing the appearance of atrophic scarring or atrophic acne scarring. In one embodiment, the compound is kynurenine for use in the treatment of atrophic scarring. In one embodiment, the compound is kynurenine for use in the treatment of atrophic acne scarring.

In one embodiment, the compound is a component of a composition formulated for injection and is administered by injection. In one embodiment the injection is administered as a subcutaneous, intradermal, or an intramuscular injection.

In one embodiment, the compound is a component of a composition formulated for oral delivery. In one embodiment the compound is an ingredient in a product intended for oral consumption.

In one embodiment, the compound is a component of a composition formulated for topical delivery. In one embodiment the compound is a component of a composition formulated for topical application. In one embodiment the compound is an ingredient in a lotion, cream, gel, solution, or suspension for topical use.

In some embodiments, the application, administration, or use of the compound is contemplated as one to five times per day. In some embodiments, the application, administration, or use of the compound is contemplated as once, twice, or thrice daily. In some embodiments, the application, administration, or use is contemplated as topical.

In one embodiment, the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid, and is a component of a composition formulated for topical use or topical application.

In one embodiment, the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid, and is a component of a composition formulated as a cream, gel, lotion, foam, suspension, or ointment.

In one embodiment, the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid, and is formulated with an additional one or more pharmaceutical composition(s), wherein said pharmaceutical compositions include a retinoid or retinoid-like compound (e.g., tretoin, adapalene, tazorotene), an antibiotic (e.g., clindamycin or erythromycin) and wherein the antibiotic may be further combined with benzoyl peroxide, azelaic acid or salicylic acid.

In one embodiment, the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid, and is formulated with an additional one or more non-prescription drugs, wherein said non-prescription drug(s) is covered by an FDA OTC monograph(s). Non-limiting examples of said non-prescription drugs include salicylic acid, benzoyl peroxide, allantoin, cocoa butter, dimethicone, glycerin, petrolatum, live yeast cell derivative (LYCD), zinc stearate, zinc acetate, zinc carbonate, zinc oxide, mineral oil, resorcinol, Peruvian balsalm, shark liver oil, and tannic acid.

In one embodiment, the compound is kynurenic acid and is between 0.1% and 1% by weight of a composition for topical use or topical application. In one embodiment, the compound is kynurenic acid and is 0.25% to 0.5% by weight of a composition for topical use or topical application. In one embodiment, the compound is kynurenic acid at 0.5% by weight of a composition for topical use or topical application. In one embodiment, the compound is kynurenic acid and is 0.5% of a lotion. In one embodiment, the compound is kynurenic acid and is 0.5% of a cream. In one embodiment, the compound is kynurenic acid and is 0.1% to 2% by weight of a composition for topical application or topical use for use in reducing the appearance of atrophic scarring, atrophic acne scarring, or post-acne atrophic scarring. In one embodiment, the compound is kynurenic acid and is 0.1% to 2% by weight of a composition for use as a cosmetic product to reduce the appearance of atrophic scarring, atrophic acne scarring, or post-acne atrophic scarring.

In one embodiment, the present invention contemplates the use of kynurenic acid in the manufacture of a cosmetic or drug product to reduce the appearance of atrophic scarring, atrophic acne scarring, or post-acne atrophic scarring. In one embodiment, the present invention contemplates a topical formulation containing a trace to 2% by weight of kynurenine, kynurenic acid, or xanthurenic acid; and the use of said formulation for reducing the appearance of atrophic scarring, atrophic acne scarring, or post-acne atrophic scarring. In one embodiment, the present invention contemplates a topical formulation containing trace to 2% by weight of kynurenine, kynurenic acid, or xanthurenic acid; and said formulation for use in reducing the appearance of atrophic scarring, atrophic acne scarring, or post-acne atrophic scarring. In one embodiment, the present invention contemplates a topical formulation containing 0.1 to 0.75% by weight of kynurenine, kynurenic acid, or xanthurenic acid; and said formulation for use in reducing the appearance of atrophic scarring, atrophic acne scarring, or post-acne atrophic scarring. In one embodiment, the present invention contemplates a topical formulation containing 0.25 to 0.5% by weight of kynurenine, kynurenic acid, or xanthurenic acid; and said formulation for use in reducing the appearance of atrophic scarring, atrophic acne scarring, or post-acne atrophic scarring. In one embodiment, the present invention contemplates a topical formulation containing 0.1 to 0.75% by weight of kynurenic acid; and said formulation for use in reducing the appearance of atrophic scarring, atrophic acne scarring, or post-acne atrophic scarring.

### IV. Examples

### A. Formulations for Topical Use

I. A topical cream was made as follows, kynurenic acid was solubilized in a phosphate buffered solution of 1 M NaOH and the pH was then adjusted to 5.5 at room temperature, this KynA solution was then added with steady mixing to a dermatological compounding base (Glaxal Base™, WellSpring, Ont., Canada) and adjusted to a pH of 6 before packaging in poly bottles. Topical cream was made at 0.15%, 0.25%, 0.4%, and 0.5% kynurenic acid by weight (Papp et al., 2018).

II. Other compounding creams are known in the art, and an example is the VersaPro Cream Base (Product #2529, MEDISCA Pharmaceutique Inc., Richmond, BC, Canada). Using the VersaPro Cream Based, dry kynurenic acid powder to make a 0.5% final weight was manually mixed into the cream using a mortar and pestle.

III. Moisturizing cream with kynurenic acid A 0.5% kynurenic acid by weight moisturizing cream was made by combining water, petrolatum, cetearyl alcohol, light mineral oil, ceteareth-20, TroyCare™ EPP37, sodium dihydrogen phosphate dihydrate, kynurenic acid, sodium hydroxide, hydrochloric acid, sodium chloride, and disodium hydrogen phosphate dihydrate. A 0.25% by weight kynurenic acid moisturizing cream was made by combining the same ingredients (water, petrolatum, cetearyl alcohol, light mineral oil, ceteareth-20, TroyCare™ EPP37, sodium dihydrogen phosphate dihydrate, kynurenic acid, sodium hydroxide, hydrochloric acid, sodium chloride, and disodium hydrogen phosphate dihydrate), wherein the amount of kynurenic acid was reduced to 0.25% of the final weight of the product.

### B. Use of Topical Kynurenic Acid (0.5%, by Weight) for Reducing Appearance of Acne Scarring

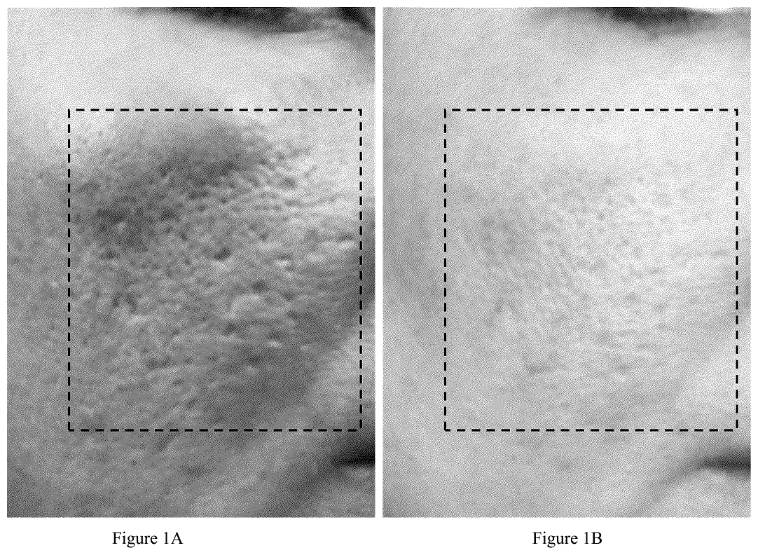

A female in their mid-40s presented with significant atrophic acne scarring on the face and self-reported that their acne scars had been treatment resistant for >20 years. The person applied a topical cream containing 0.5% kynurenic acid by weight twice daily for a 3-month period to the site of the atrophic acne scarring, an application rate of approximately 17 ug of kynurenic acid per cm² of skin per application. Initial dermatological healing can be followed by visual inspection of the site. The subject reported a significant reduction in their perceived appearance of the atrophic acne scarring starting as early as approximately 2 weeks. The subject also reported that they received unsolicited feedback from others that the subject's appearance of acne [atrophic scarring] was markedly less apparent. An illustrative image of the “before” and “during use” is shown as FIG. 1.

### C. Use of Topical Kynurenic Acid (0.5% by Weight) for Reducing the Appearance of Acne Scarring

A male of approx. 70 years of age with atrophic acne scarring on the face self-reported that the scars were “mature”, “stable”, and had persisted for “>3 years”. The person applied a topical cream containing 0.5% kynurenic acid by weight once daily for a 2-month period at an application rate of approximately 2.5-5 micrograms kynurenic acid per square cm of skin per application. The user reported that over the 2-month period, the holes and indents of his atrophic acne scars were less visible and less noticeable and appeared to be filling in. The subject also reported that others had also remarked that the user's atrophic acne scars were less visible or less noticeable.

### D. Use of Topical Kynurenine for Hypertrophic Scarring

Topical kynurenine cream at 0.05% by weight has been described in literature for use in treating hypertrophic scarring in vivo (Li et al., 2014; Poormasjedi-Meibod et al., 2014), and methods of cream manufacture and topical application are incorporated by reference.

### REFERENCES

BirchBioMed Inc. (2021, January 22). BirchBioMed Inc. Announces Positive Topline Data from Phase 2 Study of FS2 for Treatment of Keloid Scars [Press release]. Retrieved from https://www.prnewswire.com/news-releases/birchbiomed-inc-announces-positive-topline-data-from-phase-2-study-of-fs2-for-treatment-of-keloid-scars-301213375.html.
Connolly, D., Vu, H. L., Mariwalla, K., & Saedi, N. (2017). Acne Scarring-Pathogenesis, Evaluation, and Treatment Options. J Clin Aesthet Dermatol, 10(9), 12-23.
Duru, P., & Orsal, O. (2021). The effect of acne on quality of life, social appearance anxiety, and use of conventional, complementary, and alternative treatments. Complement Ther Med, 56, 102614. doi:10.1016/j.ctim.2020.102614.
Eitta, R. S. A., Ismail, A. A., Abdelmaksoud, R. A., Ghezlan, N. A., & Mehanna, R. A. (2019). Evaluation of autologous adipose-derived stem cells vs. fractional carbon dioxide laser in the treatment of post acne scars: a split-face study. Int J Dermatol, 58(10), 1212-1222. doi:10.1111/ijd.14567.
Heng, A. H. S., & Chew, F. T. (2020). Systematic review of the epidemiology of acne vulgaris. Sci Rep, 10(1), 5754. doi:10.1038/s41598-020-62715-3.
Hession, M. T., & Graber, E. M. (2015). Atrophic acne scarring: a review of treatment options. J Clin Aesthet Dermatol, 8(1), 50-58.
Li, Y., Kilani, R. T., Rahmani-Neishaboor, E., Jalili, R. B., & Ghahary, A. (2014). Kynurenine increases matrix metalloproteinase-1 and-3 expression in cultured dermal fibroblasts and improves scarring in vivo. Journal of Investigative Dermatology, 134(3), 643-650.
Papp, A., Hartwell, R., Evans, M., & Ghahary, A. (2018). The Safety and Tolerability of Topically Delivered Kynurenic Acid in Humans. A Phase 1 Randomized Double-Blind Clinical Trial. J Pharm Sci, 107(6), 1572-1576. doi:10.1016/j.xphs.2018.01.023.
Patel, L., McGrouther, D., & Chakrabarty, K. (2014). Evaluating evidence for atrophic scarring treatment modalities. JRSM Open, 5(9), 2054270414540139. doi:10.1177/2054270414540139.
Poormasjedi-Meibod, M. S., Hartwell, R., Taghi Kilani, R., & Ghahary, A. (2014). Anti-scarring properties of different tryptophan derivatives. PloS one, 9(3), e91955.
Schute D A. (2019, October 2). What Are the Most Expensive Acne Medications and How Can I Save? Retrieved from https://www.goodrx.com/blog/most-expensive-acne-meds-how-to-save/accessed Apr. 29, 2021.
Sitohang, I. B. S., Sirait, S. A. P., & Suryanegara, J. (2021). Microneedling in the treatment of atrophic scars: A systematic review of randomised controlled trials. Int Wound J. doi:10.1111/iwj.13559.

All publications, patent applications, patents, and other references mentioned herein are expressly incorporated by reference in their entirety, to the same extent as if each were incorporated by reference individually. In case of conflict, the present specification, including definitions, will control.

This invention has been described by reference to certain preferred embodiments, however, it should be understood that it may be embodied in other specific forms or variations thereof without departing from its special or essential characteristics. The embodiments described above are, therefore, considered to be illustrative in all respects and not restrictive, the scope of the invention being indicated by the appended claims rather than by the foregoing description.

We claim:

1. A method to reduce the appearance of atrophic acne scarring or post-acne atrophic scarring comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

2. A method of claim 1, wherein the small molecule compound is administered or applied as a component of a composition formulated for topical use.

3. A method of claim 2, wherein the composition is formulated as a cream.

4. A method of claim 2, wherein the compound is kynurenic acid and the composition is formulated as 0.5% kynurenic acid by weight.

5. A method of claim 2, wherein the compound is kynurenine and the composition is formulated as 0.05% kynurenine by weight.

6. A method of claim 1, wherein said administering or applying is once or twice daily.

7. A method of claim 1, wherein said administering or applying is topically to skin with atrophic scarring.

8. A method to treat atrophic acne scarring or post-acne atrophic scarring comprising of administering or applying a small molecule compound wherein the compound is selected from the group consisting of DL-kynurenine, L-kynurenine, D-kynurenine, 3-hydroxy-DL-kynurenine, 3-hydroxy-L-kynurenine, 3-hydroxy-D-kynurenine, 5-hydroxy-DL-kynurenine, 5-hydroxy-L-kynurenine, 5-hydroxy-D-kynurenine, N′-formyl-kynurenine, N-Acetyl-3-OH-kynurenine, 4-Chloro-DL-kynurenine, Xanthurenic Acid, and Kynurenic Acid.

9. A method of claim 8 wherein the small molecule compound is administered or applied as a component of a composition formulated for topical use.

10. A method of claim 9, wherein the composition is formulated as a cream.

11. A method of claim 9, wherein the compound is kynurenic acid and the composition is formulated as 0.5% kynurenic acid by weight.

12. A method of claim 9, wherein the compound is kynurenine and the composition is formulated as 0.05% kynurenine by weight.

13. A method of claim 8, wherein said administering or applying is once or twice daily.

14. A compound selected from the group consisting of kynurenine and kynurenic acid, for use in the treatment of atrophic scarring.

15. The compound for use of claim 14, wherein the atrophic scarring is atrophic acne scarring.
